(19) United States
(12) Patent Application Publication
(10) Pub. No.: US 2026/0124188 A1
(43) Pub. Date: May 7, 2026

(54) mGluR receptor antagonists for use in prevention andor treatment of bone marrow lesions (BML) andor osteoporosis, cartilage loss, osteosclerosis and osteitis in a mammal

(71) Applicant: Orphelion AB, Stockholm (SE)

(72) Inventor: Claes Thulin, Stockholm (SE)

(73) Assignee: Orphelion AB, Stockholm (SE)

(21) Appl. No.: 19/117,497

(22) PCT Filed: Oct. 4, 2023
(86) PCT No.: PCT/EP2023/077383

§ 371 Date: Apr. 1, 2025

(30) Foreign Application Priority Data
Oct. 28, 2022 (SE) 2251246-1

(51) Int. Cl. A61K 31/4439; A61K 31/135; A61K 31/44; A61K 31/444; A61K 31/4545; A61K 31/5377; A61K 31/573; A61K 31/675; A61K 31/702; A61K 31/727; A61K 38/58; A61P 19/08

(52) U.S. Cl. CPC A61K 31/4439; A61K 31/135; A61K 31/44; A61K 31/444; A61K 31/4545; A61K 31/5377; A61K 31/573; A61K 31/675; A61K 31/702; A61K 31/727; A61K 38/58; A61P 19/08

(57) ABSTRACT

The present invention relates to mGluR5 antagonists, or compounds 4-[5-[(rac)-1-[5-(3-Chlorophenyl)-3-isoxazolyl]ethoxy]-4-methyl-4H-1,2,4-triazol-3-yl]pyridine (TT00), or a pharmaceutically acceptable salt thereof, enantiomer, isotope, or mixture thereof, or a pharmaceutical composition comprising said TT00, for use in prevention and/or treatment of bone marrow lesions (BML) and/or osteoporosis, cartilage loss, osteosclerosis, osteitis, or BML related to osteoporosis, cartilage loss, osteosclerosis, osteitis, rheumatoid arthritis, osteosarcoma and/or osteoarthritis or combined with a) pain, b) GERD, c) anxiety and/or d) stress in mammals, such as humans, apes or dogs, cats, pigs, cows or horses.

### FIELD OF THE INVENTION

The present invention relates to mGluR5 antagonists, or compounds 4-[5-[(rac)-1-[5-(3-Chlorophenyl)-3-isoxazolyl]ethoxy]-4-methyl-4H-1,2,4-triazol-3-yl]pyridine (TT00), or a pharmaceutically acceptable salt thereof, enantiomer, isotope, or mixture thereof, or a pharmaceutical composition comprising said TT00, for use in prevention and/or treatment of bone marrow lesions (BML) and/or osteoporosis, cartilage loss, osteosclerosis, osteitis, or BML related to osteoporosis, cartilage loss, osteosclerosis, osteitis, rheumatoid arthritis, osteosarcoma and/or osteoarthritis or said disorders combined with a) pain, b) GERD, c) anxiety and/or d) stress in mammals, such as humans, apes or dogs, cats, pigs, cows or horses.

### BACKGROUND

Bone marrow lesions (BMLs), distinct from but originally known as ‘bone marrow edema’, can is be described as common and non-specific magnetic resonance imaging (MRI) findings associated with various pathologies. They have been most studied in knees or hips but are well-described in joints, especially joints from mammals suffering from Osteoarthritis (OA).

BML also represents microscopic collapse within the necrotic subchondral bone and indicates an unfavourable prognosis.

BMLs are characterized on MRI by ill-defined hypo intensity on T1-weighted non-fat-suppressed images, and hyperintensity on fluid-sensitive, T2-, proton density-, and intermediate-weighted fat-suppressed and short tau inversion recovery (STIR) images.

Furthermore, they enhance after intravenous administration of contrast agents.

BMLs have an array of different causes including, but not limited to,
Trauma: Fracture, local transient osteoporosis, altered stress/biomechanics (plantar fasciitis, tendinitis/enthesitis), bone bruise, and osteochondral injuries (osteochondritis dissecans)
Degenerative lesions: Osteoarthritis (hip, knee, other) and MODIC lesions (spine)
Inflammatory lesions: Inflammatory arthropathies and enthesitis (rheumatoid arthritis (RA), Ankylosing spondylitis, psoriasis) and systemic chronic inflammation with fibrosis.
Ischaemic lesions: Avascular necrosis (AVN), Complex regional pain syndrome (Sudeks atrophy of bone), Sickle cell anaemia (SCA),
Infectious lesions: Osteomyelitis, diabetic foot, Charcot foot, sepsis (bone infarcts),
Metabolic/endocrine lesions: Hydroxyapatite deposition disease (HADD), Gout
Latrogenic lesions: Surgery, Radiotherapy, immunosuppressants (glucocorticoids, cyclosporin, cytostatics) BMLs are associated with a multitude of clinical features, such as pain, progression of a disease, such as those mentioned above, transient regional osteoporosis, bone bruises or contusions, osteochondritis dissecans, increased subchondral stress, seropositive inflammatory arthropathy, osteonecrosis of the bone, complex regional pain syndrome, tissue deposition of uric acid or hydroxyapatite crystals and osteomyelitis.

There exist only a handful of treatment options for BML. Since the topic of BML is a complicated one, there is no medical consensus on the best treatment options.

One treatment option is surgery, namely, core decompression, which is a surgical procedure that involves drilling tiny holes in the damaged bone surrounding the joint. The goal is to relieve pressure and improve blood flow in the area to promote healing. As with most surgical procedures, core decompression is associated with complications, such as fractures along the core track, perforations in the femoral head, and deep vein thrombosis.

Pharmacological intervention strategies involve two types of medications that may help relieve pain in patients with BMLs. Prostacyclin is a vasodilator that promotes bone regeneration.

Bisphosphonates are used to treat multiple bone diseases because they help prevent the breakdown of bone. Existing studies show that both medications can reduce pain, improve functionality, and reduce the appearance of lesions. The possible downside is that medications only reduce or mask symptoms, but don't treat bone and joint damage. The administration of bisphosphonates is often combined with administration of (nonsteroidal anti-inflammatory drugs) NSAIDs or corticosteroids. Both NSAIDs and corticosteroids are known to have serious side effects when used over longer periods.

Glutamate is defined as an excitatory amino acid (EAA) neurotransmitter contained by the mammalian central nervous system (CNS). Evidence in literature is increasing with regard to the concept that glutamatergic signaling is also functional in non-neuronal tissues outside the CNS such as the pancreas, skin, and bone. Glutamate could have an important role in intercellular communications within bone-related cells. The involvement of EAA in peripheral nociceptive transduction has been reported in animal models suffering from acute arthritis.

Certainly, glutamate may act as a more widespread “cytokine” rather than as a “neurotransmitter” and influence a variety of cellular activities in different tissues.

The actions of extracellular glutamate are mediated by membranous receptors, which can be divided into ionotropic (iGluRs) and metabotropic (mGluRs) receptors, according to their differential intracellular signal transduction mechanisms and molecular homologies.

Metabotropic receptors are further divided into three distinct subtypes with seven transmembrane domains, including group I (mGluR1 and mGluR5), group II (mGluR2 and mGluR3), and group III (mGluR4, mGluR6, mGluR7, and mGluR8).

MGluR5 along with other receptor subtypes have been shown to mediate pain in the arthritic joint. Glutamate receptor activation may possibly be a crucial regulator in peripheral pain, chondrocyte proliferation, and immune reactions. On the other hand, glutamate receptor antagonists could potentially provide or complement novel therapies with multimodal activities against arthritis and OA symptoms. In some aspects, the mGluR5 antagonist is selected from the group comprising or consisting of TT00, MPEP, MTEP, Acamprosate, 5 Memantine, AFQ056, Fenobam, R04917523, ADX48621, ADX63365, STX107, AZD2516, Mavoglurant, LY344545, Dipraglurant, NPS 2390, (S)-MCPG, CTEP, DL-AP3 and LY341495 or a pharmaceutically acceptable salt thereof, enantiomer, isotope, or mixture thereof.

In some aspects, the mGluR5 antagonist is selected from the group comprising or consisting of TT00, MPEP, MTEP, Acamprosate, Memantine, Fenobam, R04917523, ADX48621, ADX63365, STX107, AZD2516, Mavoglurant and LY344545 or a pharmaceutically acceptable salt thereof, enantiomer, isotope, or mixture thereof.

In some aspects, the mGluR5 antagonist is selected from the group comprising or consisting of TT00, DL-AP3, CTEP (non-competitive), ABP688, (E/Z)-SIB-1893 (non-competitive), AZD 2066, MFZ 10-7, SIB-1757 (non-competitive), MFZ 10-7 hydrochloride, Mavoglurant racemate (non-competitive), AZD 2066 hydrate, MPEP Hydrochloride and MTEP Hydrochloride or a pharmaceutically acceptable salt thereof, enantiomer, isotope, or mixture thereof.

TT00, or 4-[5-[(rac)-1-[5-(3-Chlorophenyl)-3-isoxazolyl]ethoxy]-4-methyl-4H-1,2,4-triazol-3-yl]pyridine

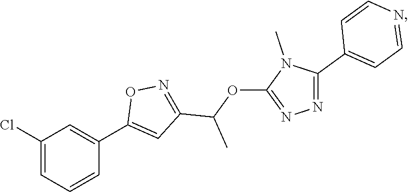 [SMILES: CC(Oc1nnc(-c2ccncc2)n1C)c1cc(-c2cccc(Cl)c2)on1]

TT002, or 4-[5-[(1S)-1-[5-(3-Chlorophenyl)-3-isoxazolyl]ethoxy]-4-methyl-4H-1,2,4-triazol-3-yl]pyridine

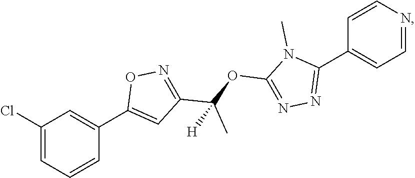

TT001, or 4-[5-[(1R)-1-[5-(3-Chlorophenyl)-3-isoxazolyl]ethoxy]-4-methyl-4H-1,2,4-triazol-3-yl]pyridine, CAS Number 934282-55-0,

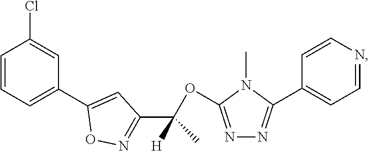 [SMILES: C[C@@H](Oc1nnc(-c2ccncc2)n1C)c1cc(-c2cccc(Cl)c2)on1]

have a molecular formula C₁₉H₁₆ClN₅O₂ and a relative molecular mass of 381.8 (base).

These compounds are selective non-competitive antagonists at metabotropic glutamate receptors subtype 5 (mGluR5) and developed for the oral treatment of chronic pain syndromes of neuropathic origin, anxiety and gastroesophageal reflux disease (GERD) in man. The developments for all indications were discontinued after clinical trials.

TT00 has an effect on the mGluR5 receptor in the central nervous system, which results in a central pain relief. Arthritis, such as osteoarthritis cause pain in the peripheral nervous system.

TT001 has been used in clinical studies. Although the development of the drug was discontinued, most clinically relevant toxicology studies have been performed on humans and animals. It is thus economically and time wise interesting to develop this compound for a new indication.

The compounds have been developed for use in prevention and treatment of pain, including peripheral pain, and stress in dogs, cats and horses. (EP3927343 and WO2021/089300), is There is an unmet need for treating bone marrow lesions (BML) and/or osteophorosis, osteoclerosis and osteitis using a safe, efficient and effective combination of drugs.

### SUMMARY OF THE INVENTION

The present invention is directed to mGluR5 antagonists, such as compounds TT00, TT001 and/or TT002, or a pharmaceutically acceptable salt thereof, enantiomer, isotope, or mixture thereof, for use in prevention and/or treatment of bone marrow lesions (BML) and/or osteoporosis, cartilage loss, osteosclerosis, osteitis, rheumatoid arthritis, osteosarcoma, in mammals. In one aspect, the compound is TT001, or a hydrochloride or sulphate salt thereof. In another aspect, the compound is a hydrochloride salt of TT001. In a further aspect, the compound is a sulphate salt of TT001. In one aspect, the mammal is a human, ape, dog, cat, pig, cow or horse. In one aspect, the mammal is a human, an ape or a dog.

The characteristics and central and peripheral working mechanism of mGluR5 antagonists, such as compounds TT00, TT001 and/or TT002 make them a very different good candidate for treatment of BML, osteoporosis, cartilage loss, osteosclerosis and/or osteitis. In some aspects, the compounds are used for prevention and/or treatment of bone marrow lesions (BML) related to osteoporosis, cartilage loss, osteosclerosis and/or osteitis. Osteoporosis, cartilage loss, osteosclerosis and osteitis are associated with inflammation, especially inflammation and edema in and around the bone that may cause lesions in the bone marrow. In some aspects, the compounds are used for prevention and/or treatment of bone marrow lesions (BML) related to rheumatoid arthritis, osteosarcoma and/or osteoarthritis, including accelerated knee osteoarthritis.

BML is associated with cartilage loss. Treatment of BML by using an MGluR5 antagonist is thus expected to have a positive effect on cartilage loss or cartilage loss related to BML or BML related to cartilage loss. In one aspect, MGluR5 antaganists are used for prevention or treatment or delay of progression of cartilage loss, or BML related to cartilage loss.

Compared to symptomatic treatment available today, it is believed that mGluR antagonists, especially mGluR5 antagonists can treat the diseases mentioned herein.

There is increasing evidence that glutamate signalling is a significant participant in bone homeostasis and disruptions in glutamatergic mechanisms can contribute to a variety of bone diseases. Glutamate transport seems significant for feedback control between bone cells (osteoblasts and osteoclasts). Disturbance of this process is believed to be relevant in osteoporosis. In osteoarthritis, glutamate receptor expression is altered in comparison to normal bone cells, and extracellular glutamate concentrations are significantly increased in is affected joints in rheumatoid arthritis and may possibly impact inflammatory responses (R: Cowan, et al., Glutamate Signaling in Healthy and Diseased Bone, Front Endocrinol (Lausanne). 10.3389/fendo.2012.00089, https://www.ncbi.nlm.nih.gov/pmc/articles/PMC3400067).

Signalling pathway inhibition has been used when there has been an upregulation of a signalling pathway. Small molecules, such as antagonists can be used to block the communication between different molecules of the pathway. These signals control many cellular processes, including growth, cell division, and death.

Since BMLs can be caused by an array of diseases containing a dysregulation in the mGluR5 pathway, the use of a mGluR5 antagonist in the prevention and treatment of BML is believed to be a valid treatment option. Even diseases, such as osteoarthritis and rheumatoid arthritis, may benefit from use of mGluR5 antagonists, because of the similar situation concerned with mGluR5. The activation of the mGluR5 pathway has also been linked to worsening diseases such as osteosarcoma (Liao S. et al., Osteosarcoma cell proliferation and survival requires mGluR5 receptor activity and is blocked by Riluzole, PLOS ONE, DOI:10.1371/journal.pone.0171256, Feb. 23, 2017). As the mGluR5 pathway is also disrupted in other bone diseases namely osteoarthritis and rheumatoid arthritis, using a mGluR5 antagonist to block the latter pathway seems a pertinent treatment option for those diseases. As BML is a combination of inflammatory diseases as well as the association of pain, which can both extensively involve the activation of the mGlur5 pathway, a mGluR5 antagonist as a treatment is believed to be effective. MGluR5 antagonists, such as compounds TT00, TT001 and/or TT002 show few side effects at the intended dose in humans, apes, dogs, cats, pigs, cows and horses. They do not lose the potency over longer administration periods. Although mGluR5 antagonists, or compounds TT00, TT001 and/or TT002 are known to affect the central nervous system, they are expected to have a positive effect in treatment of peripheral bone marrow lesions (BML) and/or osteoporosis, cartilage loss, osteosclerosis, osteitis. Such bone marrow lesions (BML) and/or osteoporosis, cartilage loss, osteosclerosis, osteitis may be chronic. TT00, TT001 and/or TT002 are believed to be effective, without incidence of any serious side effect, when used over a longer period, such as months or years. It is believed that TT00, TT001 and/or TT002 would have a reduced potential for side effects compared to conventional bone marrow lesions (BML) drugs used in mammals today, such as NSAIDS, prostacyclines or bisphosphonates, especially when treated over a longer period. The compounds are believed to have a reduced gastrointestinal and renal toxicity, especially compared to NSAIDs, in humans, apes or dogs, dogs, cats, pigs, cows and horses.

In one aspect, bone marrow lesions (BML) may be traumatic BML, i.e. associated with subchondral fracture or non-traumatic BML, such as BML associated with subchondral necrosis or osteacarcinoma or other bone (related) diseases.

In one aspect, the mGluR5 antagonist is selected from the group comprising or consisting of TT00, 2-Methyl-6-(phenylethynyl)pyridine (MPEP), 3-((2-Methyl-4-thiazolyl)ethynyl)pyridine is (MTEP), Acamprosate, Memantine, Fenobam, Basimglurant (R04917523), (4-fluorophenyl)-[(3S)-3-[3-(4-fluorophenyl)-1,2,4-oxadiazol-5-yl]piperidin-1-yl]methanone (ADX63365), 3-Fluoro-5-{5-[(2-methyl-1,3-thiazol-4-yl)ethynyl]-2-pyridinyl}benzonitrile (STX107), AZD2516, Mavoglurant, (αS)-α-Amino-α-[(1R,2R)-2-carboxycyclopropyl]-9H-xanthene-9-propanoic acid (LY344545), Dipraglurant (ADX48621), N-tricyclo[3.3.1.13,7]dec-1-yl-2-quinoxalinecarboxamide (NPS 2390), (S)-α-Methyl-4-carboxyphenylglycine ((S)-MCPG), 2-chloro-4-[2-[2,5-dimethyl-1-[4-(trifluoromethoxy)phenyl]imidazol-4-yl]ethynyl]pyridine (CTEP), DL-2-Amino-3-phosphonopropionic acid (DL-AP3), 2-[(1S,2S)-2-carboxycyclopropyl]-3-(9H-xanthen-9-yl)-D-alanine (LY341495) and [(3-fluorophenyl)methylene]hydrazone-3-fluorobenzaldehyde (DFB), N-{4-chloro-2-[(1,3-dioxo-1,3-dihydro-2H-isoindol-2-yl)methyl]phenyl}-2-hydroxybenzamide (CPPHA), (S)-(4-fluoro-phenyl)-(3-[3-(4-fluoro-phenyl)-[1,2,4]-oxadiazol-5-yl]piperidin-1-yl)methanone (ASX47273), 3-cyano-N-(1,3-diphenyl-1H-pyrazol-5-yl)benzamide (CDPPB) and 4-nitro-N-(1,3-diphenyl-1H-pyrazol-5-yl)benzamide (VU29) or a pharmaceutically acceptable salt thereof, enantiomer, isotope, or mixture thereof.

In one aspect, the mGluR5 antagonist is selected from the group comprising or consisting of 30 TT00, DL-AP3, CTEP (non-competitive), 3-[2-(6-Methyl-2-pyridinyl)ethynyl]-2-cyclohexene-1-one O-methyloxime (ABP688), (E)-2-methyl-6-(2-phenylethenyl)pyridine ((E/Z)-SIB-1893) (non-competitive), AZD2066, 3-fluoro-5-((6-methylpyridin-2-yl)ethynyl)benzonitrile (MFZ 10-7), 6-methyl-2-(phenylazo)-3-pyridinol (SIB-1757) (non-competitive), MFZ 10-7 hydrochloride, Mavoglurant racemate (non-competitive), 4-[5-[(1R)-1-[5-(3-Chlorophenyl)-3-isoxazolyl]ethoxy]-4-methyl-4H-1,2,4-triazol-3-yl]pyridine (AZD2066) hydrate, MPEP Hydrochloride and MTEP Hydrochloride or a pharmaceutically acceptable salt thereof, enantiomer, isotope, or mixture thereof.

The above mentioned mGlur5 antagonists have been proven to be selective when it comes to the inhibition of mGlur5 receptors. For this reason, they are all possible treatment options for the prevention and/or treatment of bone marrow lesions (BML) and/or osteoporosis, cartilage loss, osteosclerosis, osteitis, or BML related to osteoporosis, cartilage loss, osteosclerosis and/or osteitis or combined with a) pain, b) GERD, c) anxiety and/or d) stress in mammals, such as humans, apes or dogs, cats, pigs, cows or horses.

In one aspect, the mGluR5 antagonist is selected from the group comprising or consisting of TT00, MPEP, MTEP, Acamprosate, Memantine, Fenobam, Basimglurant, Dipraglurant, ADX63365, AZD2516, Mavoglurant and LY344545, DFB, CPPHA, ASX47273, CDPPB and VU29 or a pharmaceutically acceptable salt thereof, enantiomer, isotope, or mixture thereof. These mGluR5 antagonists that have been most extensively researched and mentioned in peer-reviewed scientific articles. The antagonists have been studied both in humans and animals with regards to multiple diseases. They have also been studied in aspects such as in pain relief which can be pertinent when it comes to the prevention and/or treatment of bone marrow is lesions (BML) and/or osteoporosis, cartilage loss, osteosclerosis, osteitis, or BML related to osteoporosis, cartilage loss, osteosclerosis and/or osteitis or combined with a) pain, b) GERD, c) anxiety and/or d) stress in mammals, such as humans, apes or dogs, cats, pigs, cows or horses. This may make these antagonists more suitable for the mentioned uses.

In one aspect, the compound is TT001, or a hydrochloride or sulphate salt thereof. In an 20 aspect, the mammal is a human, an ape, or a dog. In another aspect, the mammal is a cat, a pig, a cow or a horse.

Because the compounds work on the central nervous system, they can be used to simultaneously treat and/or prevent bone marrow lesions (BML) and/or osteoporosis, cartilage loss, osteosclerosis and osteitis and other disorders or disease.

In one aspect, the mGluR5 antagonists is used to simultaneously treat and/or prevent bone marrow lesions (BML) and/or osteoporosis, cartilage loss, osteosclerosis, osteitis or BML related to osteoporosis, cartilage loss, osteosclerosis, osteitis, rheumatoid arthritis, osteosarcoma and/or osteoarthritis and a) pain, b) GERD, c) anxiety, and/or d) stress.

The invention relates to a method of treating, preventing or reducing the risk of bone marrow 30 lesions (BML) and/or osteoporosis, cartilage loss, osteosclerosis, osteitis, or BML related to osteoporosis, cartilage loss, osteosclerosis, osteitis, rheumatoid arthritis, osteosarcoma and/or osteoarthritis or both said disorders combined with a) pain, b) GERD, c) anxiety, and/or d) stress, which comprises administering to a mammal, in need thereof, a therapeutically effective amount of one or more mGluR5 antagonists, such as compounds TT00, TT001 and/or TT002, or a pharmaceutically acceptable salt thereof, enantiomer, isotope, or mixture thereof. In one aspect, the compound is TT001, or a hydrochloride or sulphate salt thereof.

The invention further relates to a pharmaceutical composition comprising one or more mGluR5 antagonists, such as compounds TT00, TT001 and/or TT002, or a pharmaceutically acceptable salt thereof, enantiomer, isotope, or mixture thereof, in the association with a pharmaceutically acceptable adjuvant, dilutent or carrier. In one aspect, the compound is TT001, or a hydrochloride or sulphate salt thereof.

The invention also relates to a process for the preparation of a pharmaceutical composition, as defined above, which comprises mixing mGluR5 antagonists, such as compounds TT00, TT001 and/or TT002, or a pharmaceutically acceptable salt thereof, enantiomer, isotope, or mixture thereof, with a pharmaceutically acceptable adjuvant, diluent or carrier. In one aspect, the compound is TT001, or a hydrochloride or sulphate salt thereof.

One aspect relates to the pharmaceutical composition comprising mGluR5 antagonists, such as compounds TT00, TT001 and/or TT002, or a pharmaceutically acceptable salt thereof, enantiomer, isotope, or mixture thereof, in the association with a pharmaceutically acceptable adjuvant, dilutent or carrier, for use in prevention and/or treatment of bone marrow lesions (BML) and/or osteoporosis, cartilage loss, osteosclerosis, osteitis, in a mammal, such as humans, apes or dogs, cats, pigs, cows or horses. In one aspect, the compound is TT001, or a hydrochloride or sulphate salt thereof. In an aspect, the mammal is a human, an ape, or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

One aspect relates to the pharmaceutical composition, comprising mGluR5 antagonists, such as compounds TT00, TT001 and/or TT002, or a pharmaceutically acceptable salt thereof, enantiomer, isotope, or mixture thereof, in the association with a pharmaceutically acceptable adjuvant, dilutent or carrier, for use in prevention and/or treatment of both bone marrow lesions (BML) and/or osteoporosis, cartilage loss, osteosclerosis, osteitis, and a) pain, in humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

One aspect relates to the pharmaceutical composition, comprising mGluR5 antagonists, such as compounds TT00, TT001 and/or TT002, or a pharmaceutically acceptable salt thereof, enantiomer, isotope, or mixture thereof, in the association with a pharmaceutically acceptable adjuvant, dilutent or carrier, for use in prevention and/or treatment of both bone marrow lesions (BML) and/or osteoporosis, cartilage loss, osteosclerosis, osteitis, and b) gastroesophageal reflux disease (GERD) in humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

One aspect relates to the pharmaceutical composition, comprising mGluR5 antagonists, such as compounds TT00, TT001 and/or TT002, or a pharmaceutically acceptable salt thereof, enantiomer, isotope, or mixture thereof, in the association with a pharmaceutically acceptable adjuvant, dilutent or carrier, for use in prevention and/or treatment of both bone marrow lesions (BML) and/or osteoporosis, cartilage loss, osteosclerosis, osteitis, and c) anxiety in humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

One aspect relates to the pharmaceutical composition, comprising mGluR5 antagonists, such as compounds TT00, TT001 and/or TT002, or a pharmaceutically acceptable salt thereof, enantiomer, isotope, or mixture thereof, in the association with a pharmaceutically acceptable adjuvant, dilutent or carrier, for use in prevention and/or treatment of both bone marrow is lesions (BML) and/or osteoporosis, cartilage loss, osteosclerosis, osteitis, and d) stress or surmeage in humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

The invention also relates to the use of mGluR5 antagonists, such as compounds TT00, TT001 and/or TT002, or a pharmaceutically acceptable salt thereof, enantiomer, isotope, or mixture thereof, in the manufacture of a medicament for the bone marrow lesions (BML) and/or osteoporosis, cartilage loss, osteosclerosis, osteitis, or both said bone marrow lesions (BML) and/or osteoporosis, cartilage loss, osteosclerosis, osteitis combined with a) pain, b) GERD, c) anxiety, and/or d) stress, in humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

The mGluR5 antagonists, or compounds TT00, TT001 and/or TT002 may be administered at a dose of 0.1 to 5.0 mg/kg, or 0.1 to 2.0 mg/kg, or 0.1 to 1.0 mg/kg per day. One aspect relates to a dosage regime, wherein mGluR5 antagonists, or compounds TT00, TT001 and/or TT002 are administered to a mammal at a dose of 0.1 to 5.0 mg/kg once daily, or twice daily. In one aspect, the compound is TT001, or a hydrochloride or sulphate salt thereof. In an aspect, the dose is for prevention and/or treatment in a dog.

In one aspect, the dosage regime is administration of mGluR5 antagonists, or compounds TT00, TT001 and/or TT002 at a dose of 0.1 to 1.0 mg/kg once daily. In one aspect, the dosage regime is administration of mGluR5 antagonists, or compounds TT00, TT001 and/or TT002 at a dose of 0.1 to 0.5 mg/kg twice daily. In one aspect, the compound is TT001, or a hydrochloride or sulphate salt thereof.

The prevention and/or treatment of bone marrow lesions (BML) and/or osteoporosis, cartilage 5 loss, osteosclerosis, osteitis related pathology defined herein may be applied as a sole preventing and/or therapy or may involve, in addition to a mGluR5 antagonists, such as TT00, TT001 and/or TT002, conjoint treatment with conventional therapy of value in preventing and/or treating one or more disease conditions referred to herein. Such conventional therapy may include one or more of the following categories of agents: NSAIDs, anesthetics, corticosteroids, bisphosphonates, osteosarcoma drugs, anti-platelet drugs, anti-coagulant drugs, anti-thrombotic agent such as prostacyclin, and opiates.

Such conjoint treatment may be achieved by way of the simultaneous, sequential or separate dosing of the individual components of the treatment. Such combination products employ mGluR5 antagonists, such as compounds TT00, TT001 and/or TT002, or a pharmaceutically is acceptable salt thereof, enantiomer, isotope, or mixture thereof, for use in prevention and/or treatment of bone marrow lesions (BML) and/or osteoporosis, cartilage loss, osteosclerosis, osteitis in humans, apes or dogs, cats, pigs, cows or horses. In some aspects, BML is BML related to osteoporosis, cartilage loss, osteosclerosis, osteitis, rheumatoid arthritis, osteosarcoma and/or osteoarthritis.

In one aspect, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, (ii) at least one additional therapeutic agent, or a pharmaceutically acceptable salt thereof, and (iii) a pharmaceutically acceptable excipient, carrier or diluent, for use in prevention and/or treatment of bone marrow lesions (BML) and/or osteoporosis, cartilage loss, osteosclerosis and/or osteitis, in mammals, such as humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

In one aspect, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, and a pharmaceutically acceptable excipient, carrier or diluent, (ii) at least one additional therapeutic agent, or a pharmaceutically acceptable salt thereof, and a pharmaceutically acceptable excipient, carrier or diluent, for use in prevention and/or treatment of bone marrow lesions (BML) and/or osteoporosis, cartilage loss, osteosclerosis and/or osteitis, in mammals, such as humans, apes or dogs, cats, pigs, cows or horses. In some aspects, BML is BML related to osteoporosis, cartilage loss, osteosclerosis, osteitis, rheumatoid arthritis, osteosarcoma and/or osteoarthritis.

In one aspect, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, (ii) at least one additional therapeutic agent, or a pharmaceutically acceptable salt thereof, and (iii) a pharmaceutically acceptable excipient, carrier or diluent, for use in prevention and/or treatment of both bone marrow lesions (BML) and/or osteoporosis, cartilage loss, osteosclerosis and/or osteitis, and a) pain, b) GERD, c) anxiety, and/or d) stress, in mammals, such as humans, apes or dogs, cats, pigs, cows or horses. In some aspects, BML is BML related to osteoporosis, cartilage loss, osteosclerosis, osteitis, rheumatoid arthritis, osteosarcoma and/or osteoarthritis.

In one aspect, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, and a pharmaceutically acceptable is excipient, carrier or diluent, (ii) at least one additional therapeutic agent, or a pharmaceutically acceptable salt thereof, and a pharmaceutically acceptable excipient, carrier or diluent for use in prevention and/or treatment of both bone marrow lesions (BML) and/or osteoporosis, cartilage loss, osteosclerosis and/or osteitis, and a) pain, b) GERD, c) anxiety, and/or d) stress, in mammals, such as humans, apes or dogs, cats, pigs, cows or horses. In some aspects, BML is BML related to osteoporosis, cartilage loss, osteosclerosis, osteitis, rheumatoid arthritis, osteosarcoma and/or osteoarthritis.

It may be advantageous to combine TT00, TT001 and/or TT002 with an additional drug to improve the condition of the mammal. In combination with anastethics, TT00, TT001 and/or TT002 may improve the condition of the mammal, prior to, during and after a veterinary treatment, such as an operation. mGluR5 antagonists, or compounds TT00, TT001 and/or TT002 are believed to have a positive effect in GERD. It might even be possible to use mGluR5 antagonists, or compounds TT00, TT001 and/or TT002 in a combination treatment with NSAIDs as it has shown to influence GERD, which may be a serious side effect of NSAIDs. In one aspect, the compound is TT001, or a hydrochloride or sulphate salt thereof.

In one aspect, the additional therapeutic agent is an NSAID, such as butyl pyrazolidines, oxicams, propionic acid derivative, fenamic acid or coxibs. In a further aspect, NSAIDs are selected from the group comprising or consisting of oxicams, propionic acid derivatives and coxibs. In one aspect, the additional therapeutic agent is any other non-steroidal anti-inflammatory. In a further aspect, NSAIDs is an antirheumatic agent. In one aspect, the additional therapeutic agent is an opiate, such as Tramadol or Tapentadol. In one aspect, the additional therapeutic agent is an anesthetic, such as acepromazine, morphine or propofol.

In one aspect, the additional therapeutic agent is a bisphosphonate, such as zoledronic acid.

In one aspect, the additional therapeutic agent is a prostacylin.

In some aspects, more than one additional therapeutic agent is used.

In another embodiment, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, and a pharmaceutically acceptable excipient, carrier or diluent, (ii) at least one additional therapeutic agent selected from the group comprising or consisting of NSAIDs, anesthetics, corticosteroids, bisphosphonates, osteosarcoma drugs, anti-platelet drugs, anti-coagulant drugs, anti-thrombotic agent such as prostacyclin, and opiates, and a pharmaceutically acceptable excipient, carrier or diluent, for use in prevention and/or treatment of bone marrow lesions (BML) and/or osteophorosis, osteoclerosis and/or osteitis, or prevention and/or treatment of both said bone marrow lesions (BML) and/or osteoporosis, cartilage loss, osteosclerosis and/or osteitis combined with a) pain, b) GERD, c) anxiety, and/or d) stress, in a mammal, such as humans, apes or dogs, cats, pigs, cows or horses. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

In another embodiment, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, and a pharmaceutically acceptable excipient, carrier or diluent, (ii) at least one additional therapeutic agent, which is an NSAID selected from the group comprising or consisting of oxicams, propionic acid derivative, and coxibs, and a pharmaceutically acceptable excipient, carrier or diluent, for use in prevention and/or treatment of bone marrow lesions (BML) and/or osteoporosis, cartilage loss, osteosclerosis and/or osteitis, or prevention and/or treatment of both said bone marrow lesions (BML) and/or osteoporosis, cartilage loss, osteosclerosis and/or osteitis combined with a) pain, b) GERD, c) anxiety and/or d) stress in a mammal. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

In another embodiment, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, and a pharmaceutically acceptable excipient, carrier or diluent, (ii) at least one additional therapeutic agent, which is an other corticosteroids, bisphosphonates, osteosarcoma drugs and anti-thrombotic agent, non-steroidal anti-inflammatory or an antirheumatic agent, and a pharmaceutically acceptable excipient, carrier or diluent, for use in prevention and/or treatment of bone marrow lesions (BML) and/or osteoporosis, cartilage loss, osteosclerosis and/or osteitis, or prevention and/or treatment of both said bone marrow lesions (BML) and/or osteoporosis, cartilage loss, osteosclerosis and/or osteitis combined with a) pain, b) GERD, c) anxiety and/or d) stress in a mammal. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

In another embodiment, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, a pharmaceutically acceptable excipient, carrier or diluent, (ii) at least one additional therapeutic agent, which is an opiate selected from the group comprising or consisting of tramadol and tapentadol and a pharmaceutically acceptable excipient, carrier or diluent, for use in prevention and/or treatment of bone marrow lesions (BML) and/or osteoporosis, cartilage loss, osteosclerosis and/or osteitis, or prevention and/or treatment of both said bone marrow lesions (BML) is and/or osteoporosis, cartilage loss, osteosclerosis and/or osteitis combined with a) pain, b) GERD, c) anxiety and/or d) stress in a mammal. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

In another embodiment, the invention relates to a pharmaceutical composition comprising (i) mGluR5 antagonists, such as compounds TT00, TT001 and/or TT002, or a pharmaceutically acceptable salt thereof, enantiomer, isotope, or mixture thereof, a pharmaceutically acceptable excipient, carrier or diluent, (ii) at least one the additional therapeutic agent, which is an anesthetic selected from the group comprising or consisting of acepromazine, morphine or propofol, and a pharmaceutically acceptable excipient, carrier or diluent, for use in prevention and/or treatment of bone marrow lesions (BML) and/or osteoporosis, cartilage loss, osteosclerosis and/or osteitis, or prevention and/or treatment of both said bone marrow lesions (BML) and/or osteoporosis, cartilage loss, osteosclerosis and/or osteitis combined with a) pain, b) GERD, c) anxiety and/or d) stress in a mammal. In one aspect, the compound is TT001, or a hydrochloride or sulphate salt thereof. In an aspect, the mammal is a human, an ape or a dog. In another aspect, the mammal is a cat or a horse. In some aspects, BML is BML related to osteoporosis, cartilage loss, osteosclerosis, osteitis, rheumatoid arthritis, osteosarcoma and/or osteoarthritis.

Such combination products employ the mGluR5 antagonists, or compounds TT00, TT001 and/or TT002 within the dosage range described herein and the other the additional therapeutic agent(s) within approved dosage ranges and/or the dosage described in the publication reference thereof.

The pharmaceutical composition comprising mGluR5 antagonists, or compounds TT00, TT001 and/or TT002, or a pharmaceutically acceptable salt thereof, enantiomer, isotope, or mixture thereof, optionally together with one or more additional therapeutic agent as defined above, may be administered topically or orally. In one aspect, the pharmaceutical composition is administered topically. In another aspect, the pharmaceutical composition is administered intramuscularly. In a further aspect, the pharmaceutical composition is administered intravenously.

### DETAILED DESCRIPTION OF THE INVENTION

The definitions set forth in this application are intended to clarify terms used throughout this application. The term “herein” means the entire application.

It is to be understood that the expression “mGluR5 antagonists, or compounds TT00, TT001 and/or TT002” includes pharmaceutically acceptable salt thereof, diastereomer, enantiomer, isotope, pro-drug or metabolite and the like, or mixture thereof, unless specified otherwise.

As used herein, “pharmaceutically acceptable salts” refer to forms of the disclosed compounds, wherein the parent compound is modified by making acid or base salts thereof. Examples of pharmaceutically acceptable salts include, but are not limited to, mineral or organic acid salts of basic residues such as amines; alkali or organic salts of acidic residues, such as carboxylic acids; and the like. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric acid. Examples of salts are hydrochloride salts or sulphate salts, especially 4-[5-[(1R)-1-[5-(3-Chlorophenyl)-3-isoxazolyl]ethoxy]-4-methyl-4H-1,2,4-triazol-3-yl]pyridine hydrochloride or 4-[5-[(1R)-1-[5-(3-Chlorophenyl)-3-isoxazolyl]ethoxy]-4-methyl-4H-1,2,4-triazol-3-yl]pyridine sulphate.

As used herein, the phrase “or pharmaceutically acceptable salts” includes hydrates and solvates thereof.

As used herein, the phrase “BML related to osteoporosis, cartilage loss, osteosclerosis, osteitis, rheumatoid arthritis, osteosarcoma and/or osteoarthritis” means that bone marrow leasons result from or are caused by or associated with the mentioned diseases.

As used herein, the phrase “BML related to” includes “BML associated with”.

As used herein, “osteoarthritis” includes accelerated knee osteoarthritis (AKOA).

As used herein, the term “disease” is intended to include disorder, condition, disease condition or any equivalent thereof. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like diethyl ether, ethyl acetate, ethanol, isopropanol, or acetonitrile are used. mGluR5 antagonists, or compounds TT00, TT001 and/or TT002, especially TT001, may exist in particular geometric or stereoisomeric forms. The present invention takes into account all such compounds, including tautomers, R- and S-enantiomers, diastereomers, (D)-isomers, (L)-isomers, the racemic mixtures thereof, and other mixtures thereof, as being covered within the scope of this invention.

As used herein, “tautomer” means other structural isomers that exist in equilibrium resulting from the migration of a hydrogen atom. For example, keto-enol tautomerism occurs where the resulting compound has the properties of both a ketone and an unsaturated alcohol.

mGluR5 antagonists, or compounds TT00, TT001 and/or TT002, especially TT001, and salts described in this specification may be isotopically labelled (or “radio-labelled”). In that instance, one or more atoms are replaced by an atom having an atomic mass or mass number different from the atomic mass or mass number typically found in nature (i.e. naturally is occurring). Examples of suitable isotopes that may be incorporated include ²H (also written as “D” for deuterium), ³H (also written as “T” for tritium), ¹¹C, ¹³C, ¹⁴C, ¹³N, ¹⁵N, ¹⁵O, ¹⁷O, ¹⁸O, ⁸F, ³⁵S, ³⁶Cl, ⁸²Br, ⁷⁵Br, ⁷⁶Br, ⁷⁷Br, ¹²³I, ¹²⁴I, ¹²⁵s and ¹³¹I. The radionuclide that is used will depend on the specific application of that radio-labelled derivative. For example, for in vitro receptor labelling and competition assays, compounds that incorporate ³H or ¹⁴C are often useful. For radio-imaging applications ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F. The present invention includes any isotope of TT00, TT001 and/or TT002 for use in diagnosis on mammals, such as humans, apes or dogs, cats, pigs, cows or horses.

mGluR5 antagonists, or compounds TT00, TT001 and/or TT002, especially TT001, may be administered orally, parenterally, buccally, vaginally, rectally, inhalation, insufflation, sublingually, intramuscularly, subcutaneously, topically, intranasally, intraperitoneally, intrathoracically, intravenously, epidurally, intrathecally, intracerebroventricularly and by injection into the joints. Said compounds may be administered topically.

The optimum dosage and frequency of administration will depend on the particular condition being treated and its severity; the species; the age, sex, size and weight, diet, and general physical condition of the particular mammal; other medication the mammal may be taking; the route of administration; the formulation; and various other factors known to physicians and others skilled in the art.

Dosages can be readily ascertained by those skilled in the art from this disclosure and the knowledge in the art. Thus, the skilled artisan can readily determine the amount of compound and optional additives, vehicles, and/or carriers in compositions and to be administered in methods of the invention.

The mGluR5 antagonist may be selected from the group comprising or consisting of TT00, 2-Methyl-6-(phenylethynyl)pyridine (MPEP), 3-((2-Methyl-4-thiazolyl)ethynyl)pyridine (MTEP), Acamprosate, Memantine, Fenobam, Basimglurant (R04917523), (4-fluorophenyl)-[(3S)-3-[3-(4-fluorophenyl)-1,2,4-oxadiazol-5-yl]piperidin-1-yl]methanone (ADX63365), 3-Fluoro-5-{5-[(2-methyl-1,3-thiazol-4-yl)ethynyl]-2-pyridinyl}benzonitrile (STX107), AZD2516, Mavoglurant, (αS)-α-Amino-α-[(1R,2R)-2-carboxycyclopropyl]-9H-xanthene-9-propanoic acid (LY344545), Dipraglurant (ADX48621), N-tricyclo[3.3.1.13,7]dec-1-yl-2-quinoxalinecarboxamide (NPS 2390), (S)-α-Methyl-4-carboxyphenylglycine ((S)-MCPG), 2-chloro-4-[2-[2,5-dimethyl-1-[4-(trifluoromethoxy)phenyl]imidazol-4-yl]ethynyl]pyridine (CTEP), DL-2-Amino-3-phosphonopropionic acid (DL-AP3), 2-[(1S,2S)-2-carboxycyclopropyl]-3-(9H-xanthen-9-yl)-D-alanine (LY341495) and [(3-fluorophenyl)methylene]hydrazone-3-fluorobenzaldehyde (DFB), N-{4-chloro-2-[(1,3-dioxo-1,3-dihydro-2H-isoindol-2-yl)methyl]phenyl}-2-hydroxybenzamide (CPPHA), (S)-(4-fluoro-phenyl)-(3-[3-(4-fluoro-phenyl)-[1,2,4]-oxadiazol-5-yl]piperidin-1-yl)methanone (ASX47273), 3-cyano-N-(1,3-diphenyl-1H-pyrazol-5-yl)benzamide (CDPPB) and 4-nitro-N-(1,3-diphenyl-1H-pyrazol-5-yl)benzamide (VU29) or a pharmaceutically acceptable salt thereof, enantiomer, isotope, or mixture thereof.

The mGluR5 antagonist may be selected from the group comprising or consisting of TT00, MPEP, MTEP, Acamprosate, Memantine, Fenobam, Basimglurant, Dipraglurant, ADX63365, AZD2516, Mavoglurant and LY344545, DFB, CPPHA, ASX47273, CDPPB and VU29 or a pharmaceutically acceptable salt thereof, enantiomer, isotope, or mixture thereof.

The mGluR5 antagonist may be selected from the group comprising or consisting TT00, of DL-AP3, CTEP (non-competitive), 3-[2-(6-Methyl-2-pyridinyl)ethynyl]-2-cyclohexene-1-one O-methyloxime (ABP688), (E)-2-methyl-6-(2-phenylethenyl)pyridine ((E/Z)-SIB-1893) (non-competitive), AZD2066, 3-fluoro-5-((6-methylpyridin-2-yl)ethynyl)benzonitrile (MFZ 10-7), 6-methyl-2-(phenylazo)-3-pyridinol (SIB-1757) (non-competitive), MFZ 10-7 hydrochloride, Mavoglurant racemate (non-competitive), 4-[5-[(1R)-1-[5-(3-Chlorophenyl)-3-isoxazolyl]ethoxy]-4-methyl-4H-1,2,4-triazol-3-yl]pyridine (AZD2066) hydrate, MPEP Hydrochloride and MTEP Hydrochloride or a pharmaceutically acceptable salt thereof, enantiomer, isotope, or mixture thereof.

The quantity of the mGluR5 antagonists, or compounds TT00, TT001 and/or TT002, especially TT001, to be administered will vary for the dog, cat, cow, pig or horse being treated and will vary from about 0.01 ng/kg of body weight to 10 mg/kg of body weight per day. mGluR5 antagonists, or compounds TT00, TT001 and/or TT002 may be administered at a dose of 0.1 to 5.0 mg/kg, or 0.1 to 2.0 mg/kg, or 0.1 to 1.0 mg/kg per day. One aspect relates to a dosage regime, wherein mGluR5 antagonists, or compounds TT00, TT001 and/or TT002 are administered to a dog, cat or horse at a dose of 0.1 to 5.0 mg/kg once daily, or twice daily. In one aspect, the compound is TT001, or a hydrochloride or sulphate salt thereof.

In one aspect, the dosage regime is administration of mGluR5 antagonists, or compounds TT00, TT001 and/or TT002 at a dose of 0.1 to 1.0 mg/kg once daily, or 0.1 to 0.5 mg/kg twice daily, to a mammal, such as human, ape, dog, cat, pig or horse.

For preparing pharmaceutical compositions from the mGluR5 antagonists, or compounds TT00, TT001 and/or TT002, especially TT001, inert, pharmaceutically acceptable carriers can be either solid or liquid. Solid form compositions include powders, tablets, dispersible granules, capsules, cachets, and suppositories. A solid carrier can be one or more substances, which may also act as diluents, flavoring agents, solubilizers, lubricants, suspending agents, binders, or tablet disintegrating agents; it can also be an encapsulating material.

Liquid form compositions include ointments, creams, gels, aqeuous liquids, which may be formulated inside a transdermal patch.

A process for preparation of a capsule may comprise the following steps;
a) Mixing compound TT00, TT001 or TT002, especially TT001 together with additives, such as calcium hydrogen, phosphate and hydroxy propylcellulose and stir for a period, is b) Adding further additives, such as mannitol and croscarmellose sodium,
c) Adding water and granulating the mixture,
d) Drying the obtained granulate,
e) Milling the dried granulate,
f) Adding further additives, such as sodium stearyl fumarate,
g) Mixing the obtained mixture, and
h) Filling the mixture in capsules or pressing the mixture into tablets.

The pharmaceutical composition may be used in prevention and/or treatment of any disease condition or combination of conditions mentioned herein.

### Medical Use

mGluR5 antagonists, or compounds TT00, TT001 and/or TT002 are believed to be useful in the prevention and/or treatment of bone marrow lesions (BML).

Said compounds are believed to be useful in the prevention and/or treatment of osteoporosis.

Said compounds are believed to be useful in the prevention and/or treatment of osteosclerosis.

Said compounds are believed to be useful in the prevention and/or treatment of osteitis.

Said compounds are believed to be useful in the prevention and/or treatment of rheumatoid arthritis.

Said compounds are believed to be useful in the prevention and/or treatment of osteosarcoma.

Said compounds are believed to be useful in the prevention and/or treatment of cartilage loss. is Said compounds are believed to be useful in the prevention and/or treatment of osteoarthritis.

Said compounds are believed to be useful in the prevention and/or treatment of bone marrow lesions (BML) related to osteoporosis, cartilage loss, osteosclerosis and/or osteitis. Said compounds are believed to be useful in the prevention and/or treatment of bone marrow lesions (BML) related to osteoporosis.

Said compounds are believed to be useful in the prevention and/or treatment of bone marrow lesions (BML) related to rheumatoid arthritis, osteosarcoma and/or osteoarthritis.

Said medical uses are useful in mammals, such as humans, apes, dogs, cats, pigs, cows or horses. The mammal may be a human. The mammal may be a dog. The mammal may be a horse. The mammal may be a pig.

Any MGluR5 antagonist, such as those mentioned herein, may be used.

### Combination Therapies

The prevention and/or treatment of bone marrow lesions (BML) and/or osteoporosis, cartilage loss, osteosclerosis and/or osteitis, or BML related to osteoporosis, cartilage loss, osteosclerosis and/or osteitis related pathology defined herein may be applied together with conventional therapy of value in preventing and/or treating one or more disease conditions referred to herein. Such conventional therapy may include one or more of the following categories of additional therapeutic agents: NSAIDs, anesthetics, corticosteroids, bisphosphonates, osteosarcoma drugs, anti-platelet drugs, anti-coagulant drugs, anti-thrombotic agent such as prostacyclin, and opiates.

NSAIDs may be selected from the group comprising or consisting of the class of pyrazolidines that is 1,2-diphenylpyrazolidine-3,5-dione carrying a butyl group at the 4-position. It has a role as a non-narcotic analgesic, a non-steroidal anti-inflammatory drug, an antirheumatic drug, a peripheral nervous system drug, a metabolite and an EC 1.1.1.184 [carbonyl reductase (NADPH)]inhibitor. In another aspect, NSAIDs are selected from the group comprising or consisting of butyl pyrazolidines, oxicams, propionic acid derivative, fenamic acid and coxibs. The NSAIDs may be selected from the group comprising or consisting of oxicams, propionic acid derivatives and coxibs. The NSAIDs may include butyl pyrazolidines, oxicams, propionic acid derivative or coxibs. The additional therapeutic agent may be any other non-steroidal anti-inflammatory or antirheumatic agents.

Examples of opiates may include tramadol and tapentadol.

Corticosteroids may be selected from the group comprising or consisting of cortisone, prednisone, prednisolone, methylprednisolone, dexamethasone, betamethasone and hydrocortisone.

Bisphosphonates may be zoledronic acid.

Anti-thrombotic agent may be selected from the group comprising or consisting of anticoagulant agents, such as Dabigatran, Desirudin, Apixaban, Betrixaban, Edoxaban, Fondaparinux, Rivaroxaban, Heparins, Heparin, Dalteparin, Enoxaparin, Tinzaparin, Warfarin, and antiplatelet drugs, such as Aspirin, Cangrelor, Clopidogrel, Dipyridamole, Prasugrel, 20 Ticagrelor, Ticlopidine, Vorapaxar, and prostacyclin, defibrotide and anagrelide.

Osteosarcoma drugs may be selected from the group comprising or consisting of cisplatin, doxorubicin, ifosfamide, and high-dose methotrexate with leucovorin rescue.

Such conjoint treatment may be achieved by way of the simultaneous, sequential or separate dosing of the individual components for use in prevention and/or treatment of osteoporosis, cartilage loss, osteosclerosis and/or osteitis, or BML related to osteoporosis, cartilage loss, osteosclerosis, osteitis, rheumatoid arthritis, osteosarcoma and/or osteoarthritis in humans, apes, dogs, cats, pigs, cows or horses.

The combination may also comprise or consist of a pharmaceutical composition comprising (i) mGluR5 antagonists, such as compounds TT00, TT001 and/or TT002, or a pharmaceutically acceptable salt thereof, enantiomer, isotope, or mixture thereof, (ii) at least one additional therapeutic agent selected from the group consisting of NSAIDs, anesthetics, corticosteroids, bisphosphonates, osteosarcoma drugs, anti-platelet drugs, anti-coagulant drugs, anti-thrombotic agent such as prostacyclin, and opiates, and (iii) a pharmaceutically acceptable excipient, carrier or diluent.

This definition of the combination includes a pharmaceutical composition comprising (i) mGluR5 antagonists, such as compounds TT00, TT001 and/or TT002, or a pharmaceutically acceptable salt thereof, enantiomer, isotope, or mixture thereof, and a pharmaceutically acceptable excipient, carrier or diluent, and (ii) at least one additional therapeutic agent selected from the group consisting of NSAIDs, anesthetics, corticosteroids, bisphosphonates, osteosarcoma drugs, anti-platelet drugs, anti-coagulant drugs, anti-thrombotic agent such as prostacyclin, and opiates, and a pharmaceutically acceptable excipient, carrier or diluent.

Any MGluR5 antagonist, such as those mentioned herein, may be used.

The combination may be (i) TT001, or a hydrochloride or sulphate salt thereof and (ii) acepromazine.

The combination may be (i) TT001, or a hydrochloride or sulphate salt thereof and (ii) oxicams.

The combination may be (i) TT001, or a hydrochloride or sulphate salt thereof and (ii) propionic acid derivatives. The combination may be (i) TT001, or a hydrochloride or sulphate salt thereof and (ii) coxibs.

The additional therapeutic agent may be an opiate, such as tramadol and tapentadol.

The combination may be (i) TT001, or a hydrochloride or sulphate salt thereof and (ii) tramadol. The combination may be (i) TT001, or a hydrochloride or sulphate salt thereof and is (ii) tapentadol.

The additional therapeutic agent may be an anesthetic selected from the group comprising or consisting of acepromazine, morphine or propofol.

The combination may be (i) TT001, or a hydrochloride or sulphate salt thereof and (ii) acepromazine. The combination may be (i) TT001, or a hydrochloride or sulphate salt thereof and (ii) morphine. The combination may be (i) TT001, or a hydrochloride or sulphate salt thereof and (ii) propofol.

The combination may be (i) TT001, or a hydrochloride or sulphate salt thereof and (ii) corticosteroids. The combination may be (i) TT001, or a hydrochloride or sulphate salt thereof and (ii) anti-thrombotic agent. The combination may be (i) TT001, or a hydrochloride or sulphate salt thereof and (ii) prostacyline. The combination may be (i) TT001, or a hydrochloride or sulphate salt thereof and (ii) antiplatelet drugs. The combination may be (i) TT001, or a hydrochloride or sulphate salt thereof and (ii) bisphosphonates, such as zoledronic acid. The combination may be (i) TT001, or a hydrochloride or sulphate salt thereof and (ii) osteosarcoma drugs.

The combination may be for use in prevention and/or treatment of BML, osteoporosis, cartilage loss, osteosclerosis and/or osteitis, or BML related to osteoporosis, cartilage loss, osteosclerosis, osteitis, rheumatoid arthritis, osteosarcoma and/or osteoarthritis, or prevention and/or treatment of both said BML, osteoporosis, cartilage loss, osteosclerosis and/or osteitis, or BML related to osteoporosis, cartilage loss, osteosclerosis, osteitis, rheumatoid arthritis, osteosarcoma and/or osteoarthritiscombined with a) pain, b) GERD, c) anxiety, and/or d) stress.

### Pharmaceutical Composition

### Ointment

Active corresponding to TT001 Weight: 0.1 to 50 mg
propylene carbonate weight: 50 mg
paraffin, hard weight: 30 mg
beeswax white weight: 35 mg
paraffin, liquid weight: 110 mg
paraffin, white soft weight: 774.7 mg

### Cream

TT001 Weight: 0.1-100 mg
propylene glycol weight: 100 mg
isopropyl myristate weight: 50 mg
cetostearyl alcohol weight: 52.5 mg
citric acid, monohydrate (e330) weight: 0.5 mg
is disodium phosphate, anhydrous weight: 0.6 mg
water weight: a sufficient amount is added to achieve the target weight of 30 or 100 g
paraffin, liquid weight: 400 mg
macrogol cetostearyl ether weight: 7.5 mg
disodium phosphate dodecahydrate (e339) weight: 1.5 mg
imidurea weight: 2 mg

### Capsule

TABLE 1
Components and quantities for TT001 Capsules 2 mg and 8 mg

| Components | 2 mg | | 8 mg | | Function | Standard |
|---|---|---|---|---|---|---|
| TT001 | 2 | mg | 8 | mg | Drug Substance | AstraZeneca |
| Calcium hydrogen | 70.5 | mg | 68.5 | mg | Filler | Ph Eur or USP |
| phosphate dehydrate/ | | | | | | |
| Dibasic Calcium | | | | | | |
| Phosphate Dihydrate | | | | | | |
| Hydroxypropylcellulose/ | 12 | mg | 12 | mg | Binder | Ph Eur or NF |
| Hydroxypropyl Cellulose | | | | | | |
| Mannitol | 141.1 | mg | 137.1 | mg | Filler | Ph Eur or USP |
| Croscarmellose sodium | 9.6 | mg | 9.6 | mg | Disintegrant | Ph Eur or NF |
| Sodium stearyl | 4.8 | mg | 4.8 | mg | Lubricant | Ph Eur or NF |
| fumarate | | | | | | |
| Water, purified/ | q.s. | | q.s. | | Granulation | Ph Eur or USP |
| Purified water ^{a} | | | | | liquid | |
| Capsules | 1 | capsule | 1 | capsule | Capsule | JP |

### Preparation of Compounds

mGluR5 antagonists, or compounds TT00, TT001 and/or TT002 can be prepared as a free base or a pharmaceutically acceptable salt thereof by the processes described in U.S. Pat. No. 7,476,684 B2 or WO2007/040982 A1, which are hereby included by reference.

### Pharmaceutical Composition

FORMULATION METHOD FOR THE PREPARATION OF TT001 FOR I.V. DOSING, 0.1 to 10 mg/ml The formulation method is applicable at concentrations in formulation between 0.1 and 10 mg/mL corresponding to 0.262 and 26.2 μmol/mL
TT001 M.W.: 381.8 g/mol
10 The dissolution of TT001 is moderate, allow a couple of hours for complete dissolution.

### Vehicle

Preparation of 40% w/v HPβCD solution in water for injection
Excipients Hydroxypropyl-β-cyclodextrin, Kleptose, Roquette (HPβCD) 400 mg (40% w/v)
Water for injection to 1 ml (1.13 g)
is Appearance Clear
Density 1.13 g/cm³

### Formulation

Preparation of TT001 I.V. formulations between 0.1 and 10 mg/ml
TT001 parent form 0.1-10 mg
Vehicle (40% w/v HPβCD solution in water for injection) to 1 ml (1.13 g)
Appearance Clear
Density 1.13 g/cm³
Comments

The dissolution of TT001 is moderate, allow a couple of hours for complete dissolution.

### Ointment

Active corresponding to TT001 Weight: 0.1 to 50 mg
propylene carbonate weight: 50 mg
paraffin, hard weight: 30 mg
beeswax white weight: 35 mg
paraffin, liquid weight: 110 mg
paraffin, white soft weight: 774.7 mg

### Cream

TT001 Weight: 0.1-100 mg
propylene glycol weight: 100 mg
isopropyl myristate weight: 50 mg
cetostearyl alcohol weight: 52.5 mg
citric acid, monohydrate (e330) weight: 0.5 mg
disodium phosphate, anhydrous weight: 0.6 mg
water weight: a sufficient amount is added to achieve the target weight of 30 or 100 g paraffin, liquid weight: 400 mg
macrogol cetostearyl ether weight: 7.5 mg
disodium phosphate dodecahydrate (e339) weight: 1.5 mg
imidurea weight: 2 mg

### Capsule

TABLE 1
Components and quantities for TT001 Capsules 2 mg and 8 mg

| Components | 2 mg | | 8 mg | | Function | Standard |
|---|---|---|---|---|---|---|
| TT001 | 2 | mg | 8 | mg | Drug Substance | AstraZeneca |
| Calcium hydrogen | 70.5 | mg | 68.5 | mg | Filler | Ph Eur or USP |
| phosphate dehydrate/ | | | | | | |
| Dibasic Calcium | | | | | | |
| Phosphate Dihydrate | | | | | | |
| Hydroxypropylcellulose/ | 12 | mg | 12 | mg | Binder | Ph Eur or NF |
| Hydroxypropyl Cellulose | | | | | | |
| Mannitol | 141.1 | mg | 137.1 | mg | Filler | Ph Eur or USP |
| Croscarmellose sodium | 9.6 | mg | 9.6 | mg | Disintegrant | Ph Eur or NF |
| Sodium stearyl | 4.8 | mg | 4.8 | mg | Lubricant | Ph Eur or NF |
| fumarate | | | | | | |
| Water, purified/ | q.s. | | q.s. | | Granulation | Ph Eur or USP |
| Purified water ^{a} | | | | | liquid | |
| Capsules | 1 | capsule | 1 | capsule | Capsule | JP |

### Examples

### Pharmaceutical Composition

### Ointment

TT001 Weight: 0.1-100 mg
propylene carbonate weight: 50 mg
paraffin, hard weight: 30 mg
beeswax white weight: 35 mg
is paraffin, liquid weight: 110 mg
paraffin, white soft weight: 774.7 mg

### Cream

TT001 Weight: 0.1-100 mg
propylene glycol weight: 100 mg
isopropyl myristate weight: 50 mg
cetostearyl alcohol weight: 52.5 mg
citric acid, monohydrate (e330) weight: 0.5 mg
disodium phosphate, anhydrous weight: 0.6 mg
water weight: a sufficient amount is added to achieve the target weight of 30 or 100 g paraffin, liquid weight: 400 mg
macrogol cetostearyl ether weight: 7.5 mg
disodium phosphate dodecahydrate (e339) weight: 1.5 mg
imidurea Weight: 2 mg
   Measuring Effect of TT00 on Bone Marrow Lesions (BML) and/or Osteoporosis, Cartilage Loss, Osteosclerosis, Osteitis Relief Experiment

The following protocols may be used for assessment of BML and/or osteoporosis, cartilage loss, osteosclerosis, osteitis, or BML related to osteoporosis, cartilage loss, osteosclerosis and/or osteitis relief in humans, apes, cats, pigs, cows, horses or dogs.

### Methods-Murine Non-Invasive of Post Traumatic Osteoarthritis (PTOA) Animals

Twelve-week-old male C57B16 mice (˜25 g, Charles River, UK) areare randomly assigned to two groups which receive either treatment (TT001) or placebo, following a blinded protocol. Mice are randomly allocated to MB1 cages (960 cm2) in groups of 5 (12 h light-dark cycles, ad libitum food and water). Buprenorphine (0.05 mg/kg) is subcutaneously administered to the mice at the start of the experiment. The mice move freely throughout the experiment and are monitored for welfare, knee swelling (digital callipers) and lameness until the end of the experiment. Loading is performed in the morning.

Procedures are performed in compliance with regulations from the local institutional animal care and use committee.

### Induction of PTOA

Mice are first anesthetized and custom-built cups are used to hold the right ankle and knee with a flexion of 30 degree before the application of a 0.5N pre-load (ElectroForce® 3200, TA Instruments, Elstree, UK). An anterior cruciate ligament (ACL) rupture is induced by the application of a single 12N load at a velocity of 1.4 mm/s. ACL rupture is identified through the waveform as a continued increase in displacement following release of the applied compressive force with an audible “popping” sound. Cervical dislocation is used to cull the mice at multiple time points to assess for the presence of an inflammatory response and joint degeneration.

### Specimen Preparation

The hind limbs are fixed post-mortem in formalin (2 days, 10% neutral buffered formalin), decalcified for 2 weeks (4° C., 10% EDTA, Fisher Scientific, Loughborough, UK), and either embedded frontally in paraffin blocks for coronal sectioning parallel to the tibia, or embedded side on for sagittal sectioning. Serial sections (5 m) obtained at 100 m intervals through the joint, are dewaxed and rehydrated prior to staining with Haematoxylin and Eosin, Toluidine Blue or processing for immunohistochemistry. Knee joints are stored in 70% ethanol following formalin fixation for x-ray analysis. Ruptures with no obvious damage to the synovium, menisci or other joint tissues are confirmed by histology on sagittal sections and x-ray analysis for example using KODAK In-Vivo Imaging System FX Pro AMV, Lincoln, UK)23.

### Histological Scoring

The medial femoral condyle (MFC), medial tibial plateau (MTP), lateral femoral condyle (LFC), and lateral tibial plateau (LTP) from 2 to 4 coronal sections (either side of the centre of the joint, approximately 200 μm apart) are used for scoring. The latter is carried out for sub-synovial inflammation and degenerative changes by two or four independent observers, respectively, blinded to treatment. For each mouse, a single score representing the mean value from all observers and sections is used for statistical comparison. The Osteoarthritis Research Society International (OARSI) score used with Toluidine Blue stained sections (0-6 for osteoarthritic changes, 0-3 for subchondral bone changes, and 0-5 for proteoglycan depletion giving a total score out of 14 for each quadrant). In addition, the presence (+1) or absence (0) of osteophytes within the joint is noted. Haematoxylin and Eosin sections are scored for the presence of sub-synovial inflammation.)

### Immunohistochemistry

Prior to antigen retrieval (1 mg/ml trypsin for 1 h at 37° C.), consecutive coronal sections are deparaffinized and rehydrated. Each following step is performed at room temperature and between each incubation step, sections are washed 3×5 min in 0.01 M phosphate buffered saline (PBS, pH7.4) containing 0.1% (v/v) Tween*20 (wash buffer). All antibodies are diluted in wash buffer (F4/80 1:250, Cd11b 1:100, IL-6 1:500, and IL-17A 1:100). Endogenous peroxidase activity is blocked with 0.3% (v/v) hydrogen peroxide for 30 min. Sections are subsequently treated with 10% normal goat serum for 1 h prior to overnight incubation (4° C.) with primary antibody, rabbit or rat IgGs or PBS. Sections are then incubated for 30 min with biotinylated anti-rabbit or anti-rat secondary antibodies, developed with nickel enhanced diaminobenzidine (DAB) (Vectastain© Elite ABC kit, DAB, Vector Laboratories, Peterborough, UK) and lastly dehydrated, cleared in xylene and mounted. A Leica DMRB microscope is used for viewing the slides.

### Quantitative RT-PCR Analysis of Gene Expression

Four hours after ACL rupture, the knee joints directly dissected to expose the femoral condyle cartilage. Forceps are used to apply pressure to the top of the femur to “pop” the cartilage from underlying subchondral bone at the tidemark. Cartilage is pooled from injured (n=x) or uninjured (n=x) contra-lateral knees and immediately snap frozen in liquid nitrogen prior to RNA extraction using TRizol© reagent according to manufacturer's protocol (ThermoFisher Scientific). Total RNA is purified using a mirVana™ miR Isolation Kit followed by DNAse treatment (Ambion, Fisher Scientific, Loughborough, UK) following the manufacturer's protocol and assessed using a spectrophotometer (Nanodrop 1000, ThermoFisher Scientific, Stockport, UK) and 2100 Bioanalyzer (Agilent Technologies) with A260/280 values between 1.8-2.0 and RIN scores>8. Complementary DNA (cDNA, 20 μl total volume) is generated from 300 ng total RNA using SuperScript® III reverse transcriptase (Invitrogen™, Fisher Scientific, UK) and 0.5 μg random primers (Promega, Southampton, UK) according to manufacturer's instructions, and 1 μl cDNA utilized in each qPCR assay. Quantitative polymerase chain reaction (qPCR) is performed using SYBR green detection (Brilliant III Ultra-Fast SYBR© QPCR mix, Agilent Technologies) using intron-spanning primers for genes of interest (Suppl. Table S327), on a QPCR machine (MxPro3000, Agilent Technologies). All reactions are carried out at an annealing temperature of 60° C., cycling conditions are: 95° C.-3 min (1 cycle), 95° C.-15 s followed by 60° C.-30 s (40 cycles), 95° C.-1 min followed by 60° C.-30 s followed by 95° C.-30 s (1 cycle). Primers (MWG-Biotech AG, Germany), are used at a final concentration of 200 nM and validated using cDNA standard curves with all primer efficiencies between 90 and 110%.28 Data are is normalized to 18s and S3-actin which are identified from 8 reference genes using RefFinder (http://150.216.56.64/referencegene.php) as maintaining stable expression under these experimental conditions.27 Fold-change in expression of genes of interest is calculated using the 2-AACT method, after normalization to the reference genes and presented relative to the uninjured contra-lateral limb.

### Data Analysis

Data is presented as mean±standard error mean (SEM) and is tested for normality and equal variances. Moreover, data is transformed if necessary, prior to testing by general linear model analysis of variance (GLM ANOVA) and Tukey's post hoc test (Minitab 16). A p 0.05 is considered significant.

### Results

The results are expected to show that BML has been reduced or treated in the group that received TT001.

Universally, lesion regression and symptom improvement, such as decreased pain are expected following the treatment. Furthermore, with the involvement of the above treatment with the glutamate signaling pathways, an inhibition in osteoclast activity and decreased bone resorption is anticipated in the treatment groups. Previous studies have noted that the BML-linked environment shows a considerable increase in RANKL expression and other various types of osteoclast-related immune cells and proinflammatory cytokines. Taken together, treatment with TT00 is expected to limit or prevent the upregulation of those specific mediators in BML.

As BML shows a variety of different radiological occurrences, the treatment group is expected to positively display radiologic changes from non-treated patients. However, the changes in imaging findings (e.g., MRI scans) will depend on the cause of BML, which can vary from trauma to chronic inflammation. In the case of BML resulting from trauma, the findings after treatment can include decreased subchondral bone damage and/or decreased fluid build-up in the bone marrow (swelling).

Concerning the histological observations, decreases in fibrosis, necrosis, and trabecular abnormalities of the subchondral bone are expected to be observed in treatment groups.

Results from recent studies have shown that microarray comparing BML, and normal bone have a significant difference in the expression of 218 genes. Given the number of genes, it would be complicated to predict the changes in all those genes without a proper microarray analysis on the treatment groups. On the other hand, BML presents with some genes, which are highly upregulated including stathmin 2, thrombospondin 4, matrix metalloproteinase 13, and Wnt/Notch/catenin/chemokine signalling molecules that are known to be active within neuronal, osteogenic, and chondrogenic pathways. Following this reasoning and the positive effect expected from TT00, those upregulated genes can be anticipated to decrease or approach baseline levels.

Reasonably, to understand the full scope of the effects of TT00, further tests would need to be carried out.

### Animal Model of a Bone Marrow Lesion.

Bowen at al, Animal Models of bone marrow lesions in osteoarthritis, JBMR Plus, vol 6, No 3, 2022, DOI: 10.1002/jbm4.10609 describes the animal model for BML, in the subchondral bone. The rabbit has been selected as the animal of choice because it has a sufficiently large subchondral bone volume for magnetic resonance (MR) imaging, and the growth plates of the distal femur are fused at a reasonable age (6 months). Alternatively, larger animals like dogs, pigs, cars, horses, goats and cows may be used.

This model differs from known models in three ways: (i) the location of the applied load is moved from the subchondral epiphysis to a region immediately adjacent to (but not touching) articular cartilage in order to generate the BML adjacent to the subchondral bone; (ii) the location of the applied load is moved to the medial (rather than lateral) side of the joint to allow subsequent use of other means of stimulating cartilage loss (i.e. destabilization of the medial meniscus); and (iii) surgical trauma of the model is reduced by applying the loads without removing the cortex. The model has been modified so that it is possible to remove the fixture used to align the loading system at the end of the procedure, reducing postoperative discomfort to the animals and improving the quality of the MR images (which may have susceptibility artifact present due to an implanted device).

Live animal work is performed after approval by the local institutional animal care and use committee. The new anatomical location for the BML-inducing mechanical loads required a preliminary study to determine an appropriate load magnitude and number of cycles sufficient for inducing a BML. Because of the strong association between microscopic tissue damage in bone and BML size, an appropriate loading waveform should also generate microscopic tissue damage in underlying cancellous bone. Previous research found that applying mechanical loads to cancellous bone from zero to a maximum load corresponding to 2 MPa at 2 Hz for 10,000 cycles consistently generated microdamage in cancellous bone.

The same frequency and number of cycles are selected to achieve a period of loading (83 minutes) that is acceptable as determined by institutional veterinary consults while keeping the load frequency close to physiologic loading frequency (1 Hz). A preliminary study is performed using male New Zealand white rabbits, 6 to 7 months of age (Charles River Laboratories, Wilmington, MA, USA). Animals are euthanized, and cadavers are subjected to bilateral surgical insertion of an aluminum fixture on the distal femur. The fixtures are secured to the bones using 1.5-mm-diameter, 20-mm length, non-self-tapping, veterinary cortex screws (VS101.020, DePuy Synthes Vet, West Chester, PA, USA). The hindlimb is then aligned beneath a vertically mounted servo-electric materials testing device (Testbench, Bose Electroforce, Eden Prairie, MN, USA). A 6-mm-diameter cylindrical loading platen is then is inserted through the alignment hole in the fixture, and cyclic loading (zero to compression) is applied. Each limb received cyclic loading with a maximum load magnitude ranging from 5.0 to 7.5 MPa (supraphysiologic load; one load magnitude per limb, n=1-5 limbs per load magnitude). Two additional limbs are positioned within the materials testing device, but no load is applied (sham loading). After loading, the fixtures are removed, and the bones are dissected. Distal femoral heads are isolated, marrow is removed, and samples are bulk stained in calcein for 2 hours under vacuum to stain microscopic tissue damage. Distal femoral heads are then embedded undecalcified in polymethyl methacrylate and sectioned for analysis.

The percentage of joints displaying microscopic tissue damage (stained with calcein) or overt fracture of the cortical shell in any of the microscopic sections is determined. Microscopic tissue damage became apparent at a maximum load magnitude of 6.5 MPa (supraphysiologic load). Maximum load magnitudes superior to 6.5 MPa often resulted in fracture of the cortical shell. Pilot studies are performed in vivo following preliminary work in cadaver tissue. Adult male New Zealand white rabbits, 6 to 7 months of age, are used for surgery using standard techniques. Animals are intubated and anesthesia is performed through isoflurane inhalation. The right femur is subjected to sham surgery whereby the medial surfaces adjacent to the distal condyle are exposed and the aluminum alignment fixture is secured with screws. Following sham surgery, the fixture is removed, and the surgical wound is closed. Immediately after the sham surgery, the animal is repositioned, and the metallic fixture is placed on the left medial surface. The animal limb is then secured within the materials testing system and the loading platen is slowly inserted through the alignment hole until it contacted the cortex. Cyclic compressive loading is applied from zero to a load sufficient to generate microscopic tissue damage (6.5 MPa, n=2). As an additional control group, two animals are submitted to cyclic loading at a maximum load below the threshold for generation of microscopic tissue damage (5.0 MPa, n=2). After 10,000 loading cycles, the limb is removed from the materials testing system, the fixture is removed from the limb, the surgical wound is closed, and the animals are allowed to recover from anesthesia and returned to normal cage activity. Animals undergo MRI with anesthesia at 2, 3, 4, 6, and 9 weeks after surgery, or until a BML is no longer observed. Images to acquire may include: a 3-plane proton density-weighted fast spin echo series, STIR images in the axial and coronal planes, IDEAL (two-point Dixon technique) images in axial and coronal planes, and a sagittal plane T2 mapping series for quantitative evaluation of articular cartilage. Between 30 and 45 minutes is needed for total image acquisition time. Animals are allowed to recover from anesthesia and returned to cage activity.

### Results

Loaded limbs are expected to display BMLs at the location of applied mechanical loads at 2 weeks after surgery and loading. Limbs subjected to cyclic loading necessary to cause microscopic tissue damage in bone (6.5 MPa maximum load) are expected to show significantly larger BMLs than are observed in limbs experiencing lower-magnitude loads (5.0 MPa) or sham-loaded limbs.

Furthermore, the volume of BMLs in limbs submitted to the 6.5 MPa maximum load waveform is expected to continue to increase until 4 weeks after surgery/loading whereas the BMLs in limbs submitted to 5.0 MPa maximum load is expected to decrease in volume and resolved by 4 weeks after surgery/loading.

Limbs submitted to sham surgery are expected to display a very small volume of BML-like signal.

After bone marrow lesions are no longer observable, animals are euthanized, and femurs are dissected from soft tissue, fixed, decalcified, and embedded in paraffin. Coronal sections are collected and stained with hematoxylin and eosin for examination of cartilage adjacent to the location of the BML.

Histopathology and T2 mapping imaging at the end of the study are expected to show no signs of cartilage degradation in any of the limbs in this small study

These preliminary studies performed by Bowen at al demonstrate that BMLs can be generated using mechanical loading with only minimal surgical intervention (no overt fracture, no implant) and without directly damaging the overlying cartilage. Additionally, Bowens work demonstrates that BMLs generated in the absence of microscopic tissue damage in underlying cancellous bone are small in volume and resolve readily.

The results are expected to show that BML has been reduced or treated in the group that received TT001.

Universally, lesion regression and symptom improvement, such as decreased pain are expected following the treatment. Furthermore, with the involvement of the above treatment with the glutamate signaling pathways, an inhibition in osteoclast activity and decreased bone resorption is anticipated in the treatment groups. Previous studies have noted that the BML-linked environment shows a considerable increase in RANKL expression and other various types of osteoclast-related immune cells and proinflammatory cytokines. Taken together, treatment with TT00 is expected to limit or prevent the upregulation of those specific mediators in BML.

As BML shows a variety of different radiological occurrences, the treatment group is expected to positively display radiologic changes from non-treated patients. However, the changes in imaging findings (e.g., MRI scans) will depend on the cause of BML, which can vary from trauma to chronic inflammation. In the case of BML resulting from trauma, the findings after treatment can include decreased subchondral bone damage and/or decreased fluid build-up in the bone marrow (swelling).

Concerning the histological observations, decreases in fibrosis, necrosis, and trabecular abnormalities of the subchondral bone are expected to be observed in treatment groups.

Results from recent studies have shown that microarray comparing BML, and normal bone have a significant difference in the expression of 218 genes. Given the number of genes, it would be complicated to predict the changes in all those genes without a proper microarray analysis on the treatment groups. On the other hand, BML presents with some genes which are highly upregulated including stathmin 2, thrombospondin 4, matrix metalloproteinase 13, and Wnt/Notch/catenin/chemokine signalling molecules that are known to be active within neuronal, osteogenic, and chondrogenic pathways. Following this reasoning and the positive effect expected from TT00, those upregulated genes can be anticipated to decrease or approach baseline levels.

Reasonably, to understand the full scope of the effects of TT00, further tests would need to be carried out.

1. A method of preventing and/or treating bone marrow lesions (BML) and/or osteoporosis, cartilage loss, osteosclerosis, osteitis in mammals, comprising:
administering to a mammal in need thereof an effective amount of an agent selected from the group consisting of:
mGluR5 antagonists, or compounds 4-[5-[(rac)-1-[5-(3-Chlorophenyl)-3-isoxazolyl]ethoxy]-4-methyl-4H-1,2,4-triazol-3-yl]pyridine (TT00), 4-[5-[(1R)-1-[5-(3-Chlorophenyl)-3-isoxazolyl]ethoxy]-4-methyl-4H-1,2,4-triazol-3-yl]pyridine, (TT001) and 4-[5-[(1S)-1-[5-(3-Chlorophenyl)-3-isoxazolyl]ethoxy]-4-methyl-4H-1,2,4-triazol-3-yl]pyridine (TT002), and combinations thereof, or a pharmaceutically acceptable salt thereof, enantiomer, isotope, or mixture thereof.

2. The method according to claim 1, wherein the bone marrow lesions (BML) are related to osteoporosis, cartilage loss, osteosclerosis and/or osteitis.

3. The method according to claim 1, wherein the bone marrow lesions (BML) are related to rheumatoid arthritis, osteosarcoma and/or osteoarthritis.

4. The method according to claim 1, wherein the bone marrow lesions (BML) and/or osteoporosis, cartilage loss, osteosclerosis, osteitis, or BML are related to osteophorosis, osteoporasis, osteitis, rheumatoid arthritis, osteosarcoma and/or osteoarthritis and a) pain.

5. The method according to claim 1, wherein both bone marrow lesions (BML) and/or osteoporosis, cartilage loss, osteosclerosis, osteitis, or BML are related to osteoporosis, osteo clerosis, osteitis, rheumatoid arthritis, osteosarcoma and/or osteoarthritis and b) gastroesophageal reflux disease (GERD).

6. The method according to claim 1, wherein both bone marrow lesions (BML) and/or osteoporosis, cartilage loss, osteosclerosis, osteitis, or BML are related to osteoporosis, osteosclerosis, osteitis, rheumatoid arthritis, osteosarcoma and/or osteoarthritis and c) anxiety.

7. The method according to claim 1, wherein both bone marrow lesions (BML) and/or osteoporosis, cartilage loss, osteosclerosis, osteitis, or BML are related to osteoporosis, osteo clerosis, osteitis, rheumatoid arthritis, osteosarcoma and/or osteoarthritis and d) stress.

8. A pharmaceutical composition comprising mGluR5 antagonists, or compounds 4-[5-[(rac)-1-[5-(3-Chlorophenyl)-3-isoxazolyl]ethoxy]-4-methyl-4H-1,2,4-triazol-3-yl]pyridine (TT00), 4-[5-[(1R)-1-[5-(3-Chlorophenyl)-3-isoxazolyl]ethoxy]-4-methyl-4H-1,2,4-triazol-3-yl]pyridine, (TT001) and/or 4-[5-[(1S)-1-[5-(3-Chlorophenyl)-3-isoxazolyl]ethoxy]-4-methyl-4H-1,2,4-triazol-3-yl]pyridine (TT002), or a pharmaceutically acceptable salt thereof, enantiomer, isotope, or mixture thereof, in the association with a pharmaceutically acceptable adjuvant, diluent or carrier, for use in prevention and/or treatment of bone marrow lesions (BML) and/or osteoporosis, cartilage loss, osteosclerosis, osteitis, or BML related to osteophorosis, osteosclerosis, osteitis, rheumatoid arthritis, osteosarcoma and/or osteoarthritis, in mammals.

9. A pharmaceutical composition comprising (i) mGluR5 antagonists, or compounds 4-[5-[(rac)-1-[5-(3-Chlorophenyl)-3-isoxazolyl]ethoxy]-4-methyl-4H-1,2,4-triazol-3-yl]pyridine (TT00), 4-[5-[(1R)-1-[5-(3-Chlorophenyl)-3-isoxazolyl]ethoxy]-4-methyl-4H-1,2,4-triazol-3-yl]pyridine, CAS Number 934282-55-0 (TT001) and/or 4-[5-[(1S)-1-[5-(3-Chlorophenyl)-3-isoxazolyl]ethoxy]-4-methyl-4H-1,2,4-triazol-3-yl]pyridine (TT002), or a pharmaceutically acceptable salt thereof, enantiomer, isotope or mixture thereof, and a pharmaceutically acceptable excipient, carrier or diluent, and (ii) at least one additional therapeutic agent, or a pharmaceutically acceptable salt thereof, and a pharmaceutically acceptable excipient, carrier or diluent, for use in prevention and/or treatment of bone marrow lesions (BML) and/or osteoporosis, cartilage loss, osteosclerosis, osteitis, or BML related to osteoporosis, osteosclerosis, osteitis, rheumatoid arthritis, osteosarcoma and/or osteoarthritis in mammals.

10. A pharmaceutical composition comprising (i) mGluR5 antagonists, or compounds 4-[5-[(rac)-1-[5-(3-Chlorophenyl)-3-isoxazolyl]ethoxy]-4-methyl-4H-1,2,4-triazol-3-yl]pyridine (TT00), 4-[5-[(1R)-1-[5-(3-Chlorophenyl)-3-isoxazolyl]ethoxy]-4-methyl-4H-1,2,4-triazol-3-yl]pyridine, (TT001) and/or 4-[5-[(1S)-1-[5-(3-Chlorophenyl)-3-isoxazolyl]ethoxy]-4-methyl-4H-1,2,4-triazol-3-yl]pyridine (TT002), or a pharmaceutically acceptable salt thereof, enantiomer, isotope, or mixture thereof, (ii) at least one additional therapeutic agent, or a pharmaceutically acceptable salt thereof, and (iii) a pharmaceutically acceptable excipient, carrier or diluent, for use in prevention and/or treatment of bone marrow lesions (BML) and/or osteoporosis, cartilage loss, osteosclerosis, osteitis, or BML related to osteoporosis, osteosclerosis, osteitis, rheumatoid arthritis, osteosarcoma and/or osteoarthritis in mammals.

11. The pharmaceutical composition comprising (i) mGluR5 antagonists, or compounds TT00, TT001 and/or TT002 according to claim 8, for use in prevention and/or treatment of both bone marrow lesions (BML) and/or osteoporosis, cartilage loss, osteosclerosis, osteitis, or BML related to osteophorosis, osteosclerosis, osteitis, rheumatoid arthritis, osteosarcoma and/or osteoarthritis, and a) pain, b) GERD, c) anxiety and/or d) stress in mammals, such as humans, apes, dogs, cats, pigs, cows or horses.

12. The pharmaceutical composition comprising (i) mGluR5 antagonists, or compounds TT00, TT001 and/or TT002, for use according to claim 9, wherein the at least one additional therapeutic agent is selected from the group comprising NSAIDs, anesthetics, corticosteroids, bisphosphonates, osteosarcoma drugs, anti-platelet drugs, anti-coagulant drugs, anti-thrombotic agent such as prostacyclin, and opiates.

13. The pharmaceutical composition comprising (i) mGluR5 antagonists, or compounds TT00, TT001 and/or TT002 according to claim 9, wherein the at least one additional therapeutic agent is selected from the group comprising NSAIDs selected from the group comprising butyl pyrazolidines, oxicams, propionic acid derivative, fenamic acid and coxibs;
wherein the NSAIDs may be selected from the group comprising or consisting of oxicams, propionic acid derivatives and coxibs, any other non-steroidal anti-inflammatory or antirheumatic agents, opiates selected from the group comprising tramadol and tapentadol, corticosteroids selected from the group comprising cortisone, prednisone, prednisolone, methylprednisolone, dexamethasone, betamethasone and hydrocortisone, bisphosphonates such as zoledronic acid, anti-thrombotic agent selected from the group comprising anticoagulant agents, such as Dabigatran, Desirudin, Apixaban, Betrixaban, Edoxaban, Fondaparinux, Rivaroxaban, Heparins, Heparin, Dalteparin, Enoxaparin, Tinzaparin, Warfarin, and antiplatelet drugs, such as Aspirin, Cangrelor, Clopidogrel, Dipyridamole, Prasugrel, Ticagrelor, Ticlopidine, Vorapaxar, and prostacyclin, defibrotide and anagrelide, and osteosarcoma drugs selected from the group comprising cisplatin, doxorubicin, ifosfamide, and high-dose methotrexate with leucovorin rescue.

14. mGluR5 antagonists, or compounds TT00, TT001 and/or TT002 for use according to claim 1, wherein the mGluR5 antagonists is selected from the group comprising 2-Methyl-6-(phenylethynyl)pyridine (MPEP), 3-((2-Methyl-4-thiazolyl)ethynyl)pyridine (MTEP), Acamprosate, Memantine, Fenobam, Basimglurant (R04917523), (4-fluorophenyl)-[(3S)-3-[3-(4-fluorophenyl)-1,2,4-oxadiazol-5-yl]piperidin-1-yl]methanone (ADX63365), 3-Fluoro-5-{5-[(2-methyl-1,3-thiazol-4-yl)ethynyl]-2-pyridinyl}benzonitrile (STX107), AZD2516, Mavoglurant, (αS)-α-Amino-α-[(1R,2R)-2-carboxycyclopropyl]-9H-xanthene-9-propanoic acid (LY344545), Dipraglurant (ADX48621), N-tricyclo[3.3.1.13,7]dec-1-yl-2-quinoxalinecarboxamide (NPS 2390), (S)-α-Methyl-4-carboxyphenylglycine ((S)-MCPG), 2-chloro-4-[2-[2,5-dimethyl-1-[4-(trifluoromethoxy)phenyl]imidazol-4-yl]ethynyl]pyridine (CTEP), DL-2-Amino-3-phosphonopropionic acid (DL-AP3), 2-[(1S,2S)-2-carboxycyclopropyl]-3-(9H-xanthen-9-yl)-D-alanine (LY341495) and [(3-fluorophenyl)methylene]hydrazone-3-fluorobenzaldehyde (DFB), N-{4-chloro-2-[(1,3-dioxo-1,3-dihydro-2H-isoindol-2-yl)methyl]phenyl}-2-hydroxybenzamide (CPPHA), (S)-(4-fluoro-phenyl)-(3-[3-(4-fluoro-phenyl)-[1,2,4]-oxadiazol-5-yl]piperidin-1-yl)methanone (ASX47273), 3-cyano-N-(1,3-diphenyl-1H-pyrazol-5-yl)benzamide (CDPPB) and 4-nitro-N-(1,3-diphenyl-1H-pyrazol-5-yl)benzamide (VU29).

15. mGluR5 antagonists, or compounds TT00, TT001 and/or TT002 for use according to claim 1, wherein the compound is TT001, or a hydrochloride or sulphate salt thereof.

16. mGluR5 antagonists, or compounds TT00, TT001 and/or TT002 for use according to claim 1, wherein the mammal is a human.

17. mGluR5 antagonists, or compounds TT00, TT001 and/or TT002 for use according to claim 1, or the pharmaceutical composition according to any one of claims 8 to 15, wherein the mammal is a dog.

18. The pharmaceutical composition according to claim 8, wherein the mGluR5 antagonists is selected from the group comprising 2-Methyl-6-(phenylethynyl)pyridine (MPEP), 3-((2-Methyl-4-thiazolyl)ethynyl)pyridine (MTEP), Acamprosate, Memantine, Fenobam, Basimglurant (R04917523), (4-fluorophenyl)-[(3S)-3-[3-(4-fluorophenyl)-1,2,4-oxadiazol-5-yl]piperidin-1-yl]methanone (ADX63365), 3-Fluoro-5-{5-[(2-methyl-1,3-thiazol-4-yl)ethynyl]-2-pyridinyl}benzonitrile (STX107), AZD2516, Mavoglurant, (αS)-α-Amino-α-[(1R,2R)-2-carboxycyclopropyl]-9H-xanthene-9-propanoic acid (LY344545), Dipraglurant (ADX48621), N-tricyclo[3.3.1.13,7]dec-1-yl-2-quinoxalinecarboxamide (NPS 2390), (S)-α-Methyl-4-carboxyphenylglycine ((S)-MCPG), 2-chloro-4-[2-[2,5-dimethyl-1-[4-(trifluoromethoxy)phenyl]imidazol-4-yl]ethynyl]pyridine (CTEP), DL-2-Amino-3-phosphonopropionic acid (DL-AP3), 2-[(1S,2S)-2-carboxycyclopropyl]-3-(9H-xanthen-9-yl)-D-alanine (LY341495) and [(3-fluorophenyl)methylene]hydrazone-3-fluorobenzaldehyde (DFB), N-{4-chloro-2-[(1,3-dioxo-1,3-dihydro-2H-isoindol-2-yl)methyl]phenyl}-2-hydroxybenzamide (CPPHA), (S)-(4-fluoro-phenyl)-(3-[3-(4-fluoro-phenyl)-[1,2,4]-oxadiazol-5-yl]piperidin-1-yl)methanone (ASX47273), 3-cyano-N-(1,3-diphenyl-1H-pyrazol-5-yl)benzamide (CDPPB) and 4-nitro-N-(1,3-diphenyl-1H-pyrazol-5-yl)benzamide (VU29).

19. The pharmaceutical composition according to claim 8, wherein the compound is TT001, or a hydrochloride or sulphate salt thereof.

20. The pharmaceutical composition according to claim 8, wherein the mammal is a human.

21. The pharmaceutical composition according to claim 8, wherein the mammal is a dog.
